(19) United States
(12) Patent Application Publication
(10) Pub. No.: US 2021/0386430 A1
(43) Pub. Date: Dec. 16, 2021

(54) PERCUTANEOUS FILTER BALLOON FOR HEMOSTASIS IN A BIOLOGICAL VESSEL, AND A METHOD OF USING THE SAME DURING DAMAGE CONTROL SURGERY

(71) Applicant: Ashanga Yatawatta, Bullhead City, AZ (US)

(72) Inventor: Ashanga Yatawatta, Bullhead City, AZ (US)

(21) Appl. No.: 17/303,921

(22) Filed: Jun. 10, 2021

Related U.S. Application Data
(60) Provisional application No. 62/705,079, filed on Jun. 10, 2020.

(51) Int. Cl. A61B 17/12; A61F 2/01

(52) U.S. Cl. CPC A61B 17/12136; A61B 17/1204; A61B 17/12109; A61M 25/1002; A61F 2/0105; A61F 2/011; A61B 2017/12004; A61F 2/013

(57) ABSTRACT

A percutaneous procedural assembly for hemostasis in a blood vessel, and a method of using the same during damage control surgery is provided. The percutaneous procedural assembly provides a serial arrangement of a plurality of tubular segments that are selectively and independently movable between a deflated condition and an inflated condition in a consecutive and/or sequentially manner. The plurality of tubular segments define a lumen for maintaining patency, wherein a distal opening to the lumen provides a filter dimensioned to separate uncoagulated blood and coagulated blood so that upon withdrawal blood clots are removed from the blood vessel.

### CROSS-REFERENCE TO RELATED APPLICATION

This application claims the benefit of priority of U.S. provisional application No. 62/705,079, filed 10 Jun. 2020, the contents of which are herein incorporated by reference.

### BACKGROUND OF THE INVENTION

The present invention relates to medical procedures and, more particularly, a percutaneous filter balloon for hemostasis in a biological vessel, and a method of using the same during damage control surgery.

Internal bleeding from a blood vessel presents a dangerous complication of trauma. Bleeding from the inferior vena cava, which can be from trauma or even elective surgery, is usually fatal. Stopping this bleeding is extremely difficult as access to the injury site of the inferior vena cava is difficult considering the large volume blood present.

No minimally invasive methods are currently in existence to stop vena cava bleeding. Open surgery remains the only option, which is invasive and time consuming. In short, there is no method in existence to achieve sustained hemostasis of the interior of the inferior vena-cava except risky and time-consuming open exploration, which is usually too late to save lives.

As can be seen, there is a need for a percutaneous filter balloon for hemostasis in a biological vessel, and a method of using the same during damage control surgery, wherein the use of a minimally invasive percutaneous approach allows rapid and safe deployment and withdrawal from the inferior vena cava.

The filter balloon of the present invention is inflatable, wherein the inflated condition the exterior of the pressurized segments of the balloon engages and/or applies pressure to the interior walls of the blood vessel, while allowing full flow of blood within a central lumen of the filter balloon, thereby maintaining patency of the vessel. Accordingly, in the event of any holes in the vena cava, there would be little to no bleeding, affording the practitioner time and exposure to close the hole or injury site over and engaged by the filter balloon embodied of the present invention. In trauma patients, the balloon can be left inflated for few days to stop bleeding.

The distal end of the filter balloon includes a built-in filter for preventing blood clot embolization, wherein the filter material of the filter material allows blood to pass but is dimensioned to capture or catch embolization-threatening thrombus during withdrawal of the device, thereby preventing blood clots from moving up to the heart and lungs, which could result in pulmonary embolism or other trauma.

The filter balloon may be placed via the femoral vein using a percutaneous approach and can be inflated without disruption of blood flow through defining a central lumen. The present invention can be placed for short periods—for instance, for elective surgery of the liver (see below)—or the filter balloon can be kept for few days on trauma patients which allows stopping the blood loss as well as preventing blood clot embolization. The present invention can be deployed and retrieved percutaneously from the groin region of the patient.

Liver surgery is risky due to bleeding, which is difficult to control. Most of the fatal bleeding arises from the inferior vena cava, which is the largest blood vessel in the body. Extreme care is needed in liver surgery to address this problem alone and has resulted in many deaths and near-death situations.

The current solution is the exercise of extreme surgical precision and gentle techniques; however, the options in elective surgical procedures, especially in the liver of trauma patients, has been limited to application of pressure and replacement of the losses. Furthermore, current minimally invasive surgery does not afford immediate pressure application and would need expedited conversion, itself leading to poor outcomes.

As can be seen, there is a need for inferior vena cava balloon for safe liver surgery allowing minimal blood loss but maintaining patency and blood flow.

The device embodied by the present invention is an inferior vena cava balloon enabled for controlling bleeding in a segment of inferior vena cava through, in certain embodiments, completely closing off branches of the liver entering the inferior vena cava for a variable period of time during surgery. This is a minimally invasive endovascular balloon, introduced from the groin with small incision. This balloon has a large central channel, which maintains blood flow from the pelvis towards the heart with minimal disruption.

The present invention is immediately available even from a prophylactic method with the filter balloon being able to keep in place without inflation unless needed. The systemic device does not require large incisions nor systemic anti-coagulation with minimal if there are any potential complications. The systemic device is easily introduced into femoral vein at the groin or another similar vein and deployed with ultrasound or fluoroscopy guidance.

### SUMMARY OF THE INVENTION

In one aspect of the present invention, a percutaneous procedural assembly includes the following: a tubular balloon having, in an expanded condition, an exterior periphery dimensioned for encasement in a blood vessel; and the tubular balloon providing a plurality of segments consecutively arranged, wherein each of the plurality of segments is movable between a collapsed condition and an expanded condition, and wherein each of the plurality of segments is fluidly coupled in such a way as to be independently expandable relative to remaining segments of the plurality of segments.

In another aspect of the present invention, the percutaneous procedural assembly may include the following: wherein the tubular balloon defines a central lumen communicated to two openings on each end of the tubular balloon; a filter covering a distal opening of the tubular balloon, wherein the filter is dimensioned to separate coagulated and uncoagulated blood, wherein the filter protrudes beyond a distal end of the tubular balloon, wherein the filter defines a tapered shape, wherein the filter is an elastic body of a stiffness to resist deformation of an applied force of pressurized blood flow; an elongated guide extending from a proximal end of the tubular balloon; and a sheath operatively associated with the tubular balloon for introduction into the blood vessel in such a way that the sheath is slidably along the elongated guide.

In yet another aspect of the present invention, a method of percutaneously treating or preventing damage to an inferior vena cava of a patient includes the following: providing the above-mentioned percutaneous procedural assembly; placing an elongated guide in the inferior vena cava; placing a sheath over the elongated guide and said percutaneous procedural assembly; positioning said percutaneous procedural assembly in the inferior vena cava; and sequentially moving the plurality of segments to the inflated condition; and thereafter sequentially moving the plurality of segments to the deflated condition; and withdrawing said percutaneous procedural assembly from a groin region of the patient wherein coagulated blood is caught by the filter of said percutaneous procedural assembly.

These and other features, aspects and advantages of the present invention will become better understood with reference to the following drawings, description and claims.

### BRIEF DESCRIPTION OF THE DRAWINGS

FIG. 1 is a schematic view of an exemplary embodiment of a procedural assembly (including the segmented balloon, a sheath, and a guide wire) of the present invention;

FIG. 2 is a schematic view of an exemplary embodiment of the segmented filter balloon of the present invention, shown in use;

FIG. 3 is a schematic section view of an exemplary embodiment of the present invention, shown in use;

FIG. 4 is a bottom perspective view of an exemplary embodiment of the segmented filter balloon device of the present invention;

FIG. 5 is a top perspective view of an exemplary embodiment of the segmented filter balloon device of the present invention; and

FIG. 6 is a flow chart of an exemplary embodiment of the present invention.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, an embodiment of the present invention provides a percutaneous procedural assembly for hemostasis in a blood vessel, and a method of using the same during damage control surgery is provided. The percutaneous procedural assembly provides a serial arrangement of a plurality of tubular segments that are selectively and independently movable between a deflated condition and an inflated condition in a consecutive and/or sequentially manner. The plurality of tubular segments define a lumen for maintaining patency, wherein a distal opening to the lumen provides a filter dimensioned to separate uncoagulated blood and coagulated blood so that upon withdrawal blood clots are removed from the blood vessel.

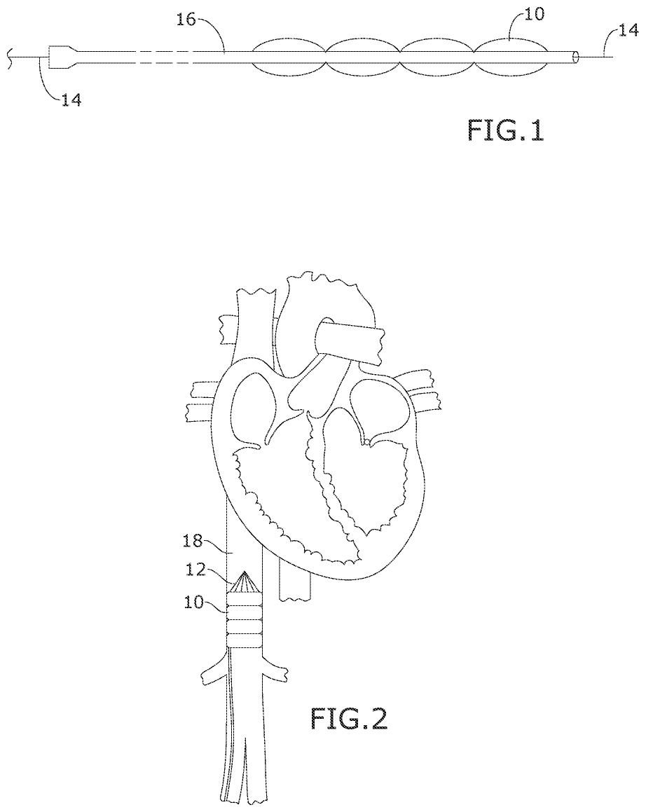

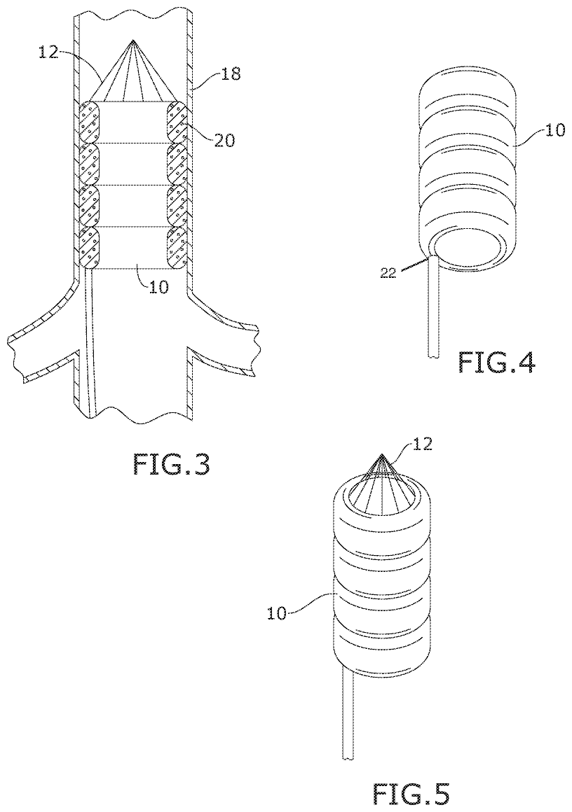

Referring now to FIGS. 1 through 6, the present invention may include a percutaneous filter balloon 10. The filter balloon 10 may include a plurality of inflatable segments 20 disposed in series in such a way as to define a lumen extending the length of the filter balloon 10. The plurality of inflatable segments 20 may be fluidly connected in both series and parallel so as to be inflatable in sequence, yet each segment 20 is adapted to maintain sufficient air pressure if an adjacent segment 20 leaks or is ruptured. In certain embodiments, each of the plurality of segments 20 is movable between a deflated condition and an inflated condition, and wherein each of the plurality of segments is fluidly coupled to a fluid inlet 22 in such a way to be independently fluidly pressurized sequentially relative to remaining segments of the plurality of segments.

A distal segment 20 of the filter balloon 10 may provide a filter 12. The filter 12 may protrude forward of the filter balloon 10 in the inflated condition based on the connection of the filter 12 to the distal-most segment 20/filter balloon 10. The filter 12 may taper to a point. The filter 12 may be dimensions, and adapted, and composed of a filter medium through which fluid blood can pass but thrombus or coagulated blood cannot pass.

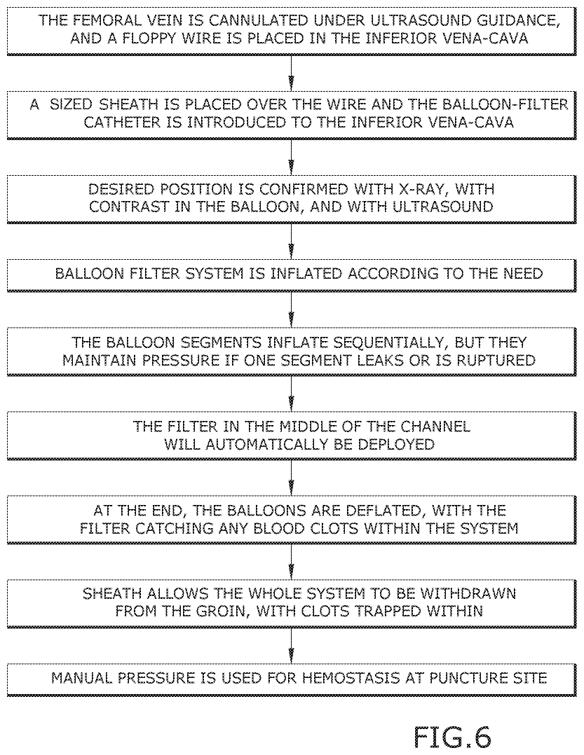

Referring the FIG. 6, a method of employing the percutaneous filter balloon 10 may include the following. The femoral vein may be cannulated under ultrasound guidance and a floppy guide/wire 14 may be placed in the inferior vena-cava 14. The guide 14 may be elongated—i.e., having a length substantially longer than a width/diameter. An appropriately sized sheath (including but not limited to a size 7 sheath) 16 may be placed over the wire 14 and the filter balloon 10 catheter in a deflated condition (see FIG. 1) may be introduced to the inferior vena-cava. Desired position is confirmed with x-ray with contrast in the balloon and with ultrasound. In situ, the plurality of segments 20 may be selectively inflated according to need. The filter in the middle of the channel will automatically be deployed. Manual pressure may be used for hemostasis at puncture site. At the end, the balloon segments are deflated with the filter 12 catching any blood clots within the system/lumen. The sheath 16 allows the whole systems to be withdrawn from the groin, with clots trapped within.

The catheter may be made from medical grade plastics, with the balloon segments 20 having high compliance and the filter 12 may be made from wiry material having the requisite stiffness as a function of the wire's elastic modulus when applied to the force of a flow of blood under the highest blood pressures possible in a human patient. The filter 12 can be optional for short term use. Balloon segments 20 will have ability to be inflated individually or all at once. The filter balloon 10 may be modified to suit other areas of the body such as thoracic or iliac veins. The length of the balloon, diameter and stiffness may be changed according to the intended target and purpose.

The present invention can be used in at least two situations. First, in elective surgery with high risk of damage to the inferior vena-cava, the filter balloon 10 may be prophylactically placed before the surgery, reducing the risk of bleeding should there be any injury. Repair of such injuries can be performed with clear view and without time restraints.

Second, in trauma situations, the filter balloon 10 can be placed and left in placed with the risk of ongoing bleeding as well as the risk of blood clot embolization is substantially reduced. In accordance with damage control surgery, the system can be removed easily once the acute phase has resolved. There would be no need to blood thinners during this period.

Additionally, a modified balloon filter catheter can be placed into any vein with risk of bleeding, such as in the thorax of the pelvis with similar approach. For instance, an inferior vena cava balloon for safe liver surgery allowing minimal blood loss but maintaining patency and blood flow as the systemic unibody device provides a balloon and filter built into a catheter and contained within a sheath.

The balloon 10 may be inflated with a hub and introduced via the access vein site after wire placement to the target site under fluoroscopy. The balloon is visible with ultrasound and fluoroscopy. Removal is performed with deflation of the balloon with aspiration at the port and pulling back into the sheath. The systemic device may be deployed from the access vein with a small incision under ultrasound guidance-ultrasound and/or fluoroscopy can be used to deploy the balloon within the catheter into a desired location. For example, the systemic unibody device may be ultrasound guided when accessing the femoral vein by way of a micro-puncture method, wherein a floppy (0.035″) wire 14 may be placed into desired location, such as retro-hepatic inferior vena cava. Then the balloon catheter contained within the sheath 16 may be introduced over wire to the desired location. Then the wire 14 may be removed and balloon catheter carefully unsheathed under imaging.

Unsheathing of the balloon may be done by pulling back the sheath 16 and under imaging, the balloon 10 may be slowly inflated with dilute contrast to approximate the balloon wall to the inferior vena cava. The removal starts with deflation of the balloon. Any clots trapped within the filter will remain with the balloon. The sheath may be pushed up into the balloon to re-sheath and contain the balloon. Clots may prevent full ensheathment but will still allow withdrawal regardless. The sheath with balloon is pulled back slowly together and pressure applied to opening until hemostasis.

Diluted contrast may be used to inflate the balloon till fully approximated against the wall of the inferior vena cava. The size for full inflation may be measured from preoperative cross-sectional imaging as well as intra-operative venograms.

The filter within the balloon catheter may be deployed with the inflation of the balloon and constrained with deflation. Any clots within the filter can be pulled back partially or completely concealed within the balloon with the sheath.

The opening made in the access vein can easily be closed with firm pressure after removal of the device.

This device with the balloon and filter will be made with medical grade plastics consistent with similar endovascular devices with anti-thrombotic properties, flow optimization and clot entrapment as core properties. The sheath will be a standard size sheath to contain the constrained device. The balloon will be interconnected with segments to allow more protection in the event of puncture of one segment, allowing other segments still being functional.

As mentioned above, other locations and access veins can be used with a comparable methodology. Additionally, the present invention can be used on trauma patients with retroperitoneal to achieve hemostasis. Thoracic great vessel bleeding or anticipated bleeding can be protected in a similar manner. The systemic devices can also be used for pelvic veins as well as the thoracic vessels. The systemic device can also be used for any other surgical procedure such as kidney, adrenal and retro-peritoneal surgery as well as trauma patients with bleeding from retro-peritoneal or the pelvic region with or without surgical or embolization options.

It should be understood, of course, that the foregoing relates to exemplary embodiments of the invention and that modifications may be made without departing from the spirit and scope of the invention as set forth in the following claims.

What is claimed is:

1. A percutaneous procedural assembly, comprising:
a tubular balloon having, in an expandable condition, an exterior periphery dimensioned for encasement in a blood vessel; and
the tubular balloon providing a plurality of segments consecutively arranged, wherein each of the plurality of segments is movable between a collapsed condition and an expanded condition, and wherein each of the plurality of segments is operatively coupled in such a way as to be independently expandable relative to remaining segments of the plurality of segments.

2. The percutaneous procedural assembly of claim 1, wherein the tubular balloon defines a central lumen communicated to two openings on each end of the tubular balloon.

3. The percutaneous procedural assembly of claim 1, further comprising a filter covering a distal opening of the tubular balloon, wherein the filter is dimensioned to separate coagulated and uncoagulated blood.

4. The percutaneous procedural assembly of claim 3, wherein the filter protrudes beyond a distal end of the tubular balloon.

5. The percutaneous procedural assembly of claim 4, wherein the filter defines a tapered shape.

6. The percutaneous procedural assembly of claim 4, wherein the filter is an elastic body of a stiffness to resist deformation of an applied force of pressurized blood flow.

7. The percutaneous procedural assembly of claim 6, further comprising an elongated guide extending from a proximal end of the tubular balloon.

8. The percutaneous procedural assembly of claim 7, further comprising a sheath operatively associated with the tubular balloon for introduction into the blood vessel in such a way that the sheath is slidably along the elongated guide.

9. A method of percutaneously treating or preventing damage to an inferior vena cava of a patient, the method comprising:
providing the percutaneous procedural assembly of claim 6;
placing an elongated guide in the inferior vena cava;
placing a sheath over the elongated guide and said percutaneous procedural assembly;
positioning said percutaneous procedural assembly in the inferior vena cava; and
sequentially moving the plurality of segments to the expandable condition.

10. The method of claim 9, further comprising:
sequentially moving the plurality of segments to the collapsed condition; and
withdrawing said percutaneous procedural assembly from a groin region of the patient wherein coagulated blood is caught by the filter of said percutaneous procedural assembly.
